(19) United States
(12) Patent Application Publication
(10) Pub. No.: US 2015/0238412 A1
(43) Pub. Date: Aug. 27, 2015

(54) ADMINISTRATION OF DIHYDROERGOTAMINE MESYLATE PARTICLES USING A METERED DOSE INHALER

(71) Applicant: MAP Pharmaceuticals, Inc., Irvine, CA (US)

(72) Inventors: Donald J. Kellerman, San Jose, CA (US); Shashidhar H. Kori, Mountain View, CA (US); Scott W. Borland, Santa Clara, CA (US); Thomas A. Armer, Cupertino, CA (US)

(21) Appl. No.: 14/699,797

(22) Filed: Apr. 29, 2015

Related U.S. Application Data
(63) Continuation of application No. 12/823,981, filed on Jun. 25, 2010.
(60) Provisional application No. 61/269,688, filed on Jun. 26, 2009.

(51) Int. Cl. A61K 9/00; A61K 31/48

(52) U.S. Cl. CPC A61K 9/008; A61K 31/48

(57) ABSTRACT

Disclosed are compositions of matter and related methods that provide a metered dose inhaler that includes a formulation having a dose that comprises a hydrofluoroalkane propellant and dihydroergotamine mesylate particles; wherein the dose includes between 0.1 mg to 4 mg of dihydroergotamine mesylate; wherein the dihydroergotamine mesylate particles have a cumulative drug substance particle size distribution with d10>0.5 micron volumetric median diameter and d90<5.0 micron volumetric median diameter.

### CROSS REFERENCE TO RELATED CASES

This application claims the benefit of U.S. Provisional Patent Application Ser. No. 61/269,688, filed on Jun. 26, 2009, the content of which is hereby incorporated by reference in its entirety.

### TECHNICAL FIELD OF THE INVENTION

The invention relates to treatments and compositions for treating headaches, and more particularly to treating subjects experiencing headaches with dihydroergotamine mesylate without contributing to the development of a fibrotic condition in the subject.

### BACKGROUND OF THE INVENTION

Headache is a fairly common indication that ranges in severity from fairly mild and transitory to dehabilitating and chronic in duration. Headaches can have significant impact on individuals and society in aggregate.

Severe headaches, such as migraine, can be fairly common. For instance acute migraine affects approximately 13% of the population, predominately in females. See R B Lipton et al. “Migraine in the United States: a review of epidemiology and health care use.” Neurology 43 (6 Suppl 3): S6-10 (1993); B K Rasmussen et al. (1992). “Migraine with aura and migraine without aura: an epidemiological study.” Cephalalgia 12 (4): 221-8 (1992); T J Steiner et al. “The prevalence and disability burden of adult migraine in England and their relationships to age, gender and ethnicity”. Cephalalgia 23 (7): 519-27. (2003); M E Bigal et al. “Age-dependent prevalence and clinical features of migraine”. Neurology 67 (2): 246-51 (2006).

Ergot derivatives have been used for treatment of various kinds of headaches, including migraines. In particular, salts of ergotamine and dihydroergotamine have been used in headache treatments. See Raskin, Neurology 36:995-997 (1986). Also see J Olesen et al. eds. The Headaches, 2nd edn. Philadelphia: Lippincott Williams & Wilkins (2000.) While conventionally shown to be effective against headaches including migraine through intravenous routes of administration, ergot derivatives have also been linked to certain adverse events including fibrosis that have limited their use.

Fibrosis is the formation or development of excess fibrous connective tissue in an organ or tissue as a repairing or reactive process, as opposed to a formation of fibrous tissue as a normal constituent of an organ or tissue. Fibrosis of the lungs and heart has been noted in patients treated with ergot derivatives. Retroperitoneal fibrosis and valvulopathy are the commonest forms of fibrosis, although pleuropulmonary fibrosis has also been seen clinically. Pfitzenmeyer et al, reported on pleuropulmonary changes induced by chronic administration of ergoline compounds used to treat Parkinsonianism and migraine. (P. Pfitzenmeyer, et. al, “Pleuropulmonary changes induced by ergoline drugs,” Eur Respir J, 9, 1013-1019, 1996.) Malaquin et al. reported pleural and retroperitoneal fibrosis in a patient who had been administered DHE orally and by injection. Malaquin et al., “Pleural and Retroperitoneal Fibrosis from Dihydroergotamine,” N. Engl. J. Med 1760 (1989.) Roth has postulated generally that compounds which agonize serotonin receptor 5HT_{2b} such as the ergoline drugs, including ergotamines and dihydroergotamine can induce valvular heart disease. (B. L. Roth, “Drugs and Valvular Heart Disease,” N Engl. J Med. 356:6-9 2007.)

Accordingly, methods and compositions are needed that allow the use of ergoline derivatives to treat headaches in a subject without contributing to the development of a fibrotic condition in the subject.

### SUMMARY OF THE INVENTION

In an aspect, the invention relates to a composition of matter comprising: a metered dose inhaler that comprises a formulation that comprises a dose that comprises a hydrofluoroalkane propellant and dihydroergotamine mesylate particles; wherein the dose comprises between 0.1 mg to 4 mg of dihydroergotamine mesylate; wherein the dihydroergotamine mesylate particles have a cumulative drug substance particle size distribution with d10>0.5 micron volumetric median diameter and d90<5.0 micron volumetric median diameter; wherein d10 is defined as the point on a cumulative volume percent distribution curve wherein 10% of the dihydroergotamine mesylate particles have a smaller volumetric median diameter; wherein d90 is defined as the point on a cumulative volume percent distribution curve wherein 90% of the dihydroergotamine mesylate particles have a smaller volumetric median diameter; and wherein volumetric diameter is as measured by laser diffraction sizing.

In another aspect, the invention relates to a method comprising: providing a metered dose inhaler that comprises a formulation that comprises a dose that comprises a hydrofluoroalkane propellant and dihydroergotamine mesylate particles; and administering the formulation to a subject by oral inhalation; wherein the dose comprises between 0.1 mg to 4 mg of dihydroergotamine mesylate; wherein the dihydroergotamine mesylate particles have a cumulative drug substance particle size distribution with d10>0.5 micron volumetric median diameter and d90<5.0 micron volumetric median diameter; wherein d10 is defined as the point on a cumulative volume percent distribution curve wherein 10% of the dihydroergotamine mesylate particles have a smaller volumetric median diameter; wherein d90 is defined as the point on a cumulative volume percent distribution curve wherein 90% of the dihydroergotamine mesylate particles have a smaller volumetric median diameter; and wherein volumetric diameter is as measured by laser diffraction sizing.

In yet other aspects, the dose of dihydroergotamine mesylate may range from 0.05 mg to 4 mg, 0.05 mg to 3.5 mg, 0.05 mg to 3.0 mg, 0.05 to 2.5 mg, 0.05 to 2 mg, 0.05 mg to 1.5 mg, 0.05 mg to 1.0 mg, or 0.05 to 0.1 mg. In some instances, the dose of dihydroergotamine mesylate may be 0.05 mg, 0.1 mg, 0.5 mg, 1.0 mg, 1.5 mg, 2.0 mg, 2.5 mg, 3.0 mg, 3.5 mg, or 4 mg.

In yet another aspect, the invention relates to a method comprising: providing to a subject a metered dose inhaler that comprises a formulation that comprises a hydrofluoroalkane propellant and dihydroergotamine mesylate particles; and informing the subject or a health care worker that administration, using the metered dose inhaler, of a dose of the formulation that comprises between 0.1 mg to 4 mg of dihydroergotamine mesylate does not contribute to the development of a fibrotic condition in the subject.

### BRIEF DESCRIPTION OF THE DRAWING

FIG. 1 shows an inhalation apparatus used in Example 2.

### DETAILED DESCRIPTION OF THE INVENTION

### Introduction

The inventors have found, surprisingly, that the problems noted in the art can be addressed by providing compositions of matter, along with related methods that comprise a metered dose inhaler that comprises a formulation that comprises a dose that comprises a hydrofluoroalkane propellant and dihydroergotamine mesylate particles; wherein the dose comprises between 0.1 mg to 4 mg of dihydroergotamine mesylate; wherein the dihydroergotamine mesylate particles have a cumulative drug substance particle size distribution with d10>0.5 micron volumetric median diameter and d90<5.0 micron volumetric median diameter; wherein d10 is defined as the point on a cumulative volume percent distribution curve wherein 10% of the dihydroergotamine mesylate particles have a smaller volumetric median diameter; wherein d90 is defined as the point on a cumulative volume percent distribution curve wherein 90% of the dihydroergotamine mesylate particles have a smaller volumetric median diameter; and wherein volumetric diameter is as measured by laser diffraction sizing.

Additionally, the problems noted in the art can be addressed by method comprising: providing a metered dose inhaler that comprises a formulation that comprises a dose that comprises a hydrofluoroalkane propellant and dihydroergotamine mesylate particles; and administering the formulation to a subject by oral inhalation; wherein the dose comprises between 0.1 mg to 4 mg of dihydroergotamine mesylate; wherein the dihydroergotamine mesylate particles have a cumulative drug substance particle size distribution with d10>0.5 micron volumetric median diameter and d90<5.0 micron volumetric median diameter; wherein d10 is defined as the point on a cumulative volume percent distribution curve wherein 10% of the dihydroergotamine mesylate particles have a smaller volumetric median diameter; wherein d90 is defined as the point on a cumulative volume percent distribution curve wherein 90% of the dihydroergotamine mesylate particles have a smaller volumetric median diameter; and wherein volumetric diameter is as measured by laser diffraction sizing.

Further, the problems noted in the art can be addressed by a method comprising: providing to a subject a metered dose inhaler that comprises a formulation that comprises a hydrofluoroalkane propellant and dihydroergotamine mesylate particles; and informing the subject or a health care worker that administration, using the metered dose inhaler, of a dose of the formulation that comprises between 0.1 mg to 4 mg of dihydroergotamine mesylate does not contribute to the development of a fibrotic condition in the subject.

In particular, the inventors noted that, despite the conventional understanding that certain ergot derivatives may contribute to elevated levels of fibrosis, this was not observed in extensive animal and human trials. In particular, as shown in Examples 2 and 3, the administration of dihdroergotamine particles having a cumulative drug substance particle size distribution with d10>0.5 micron volumetric median diameter and d90<5.0 micron volumetric median diameter did not result in elevated levels of fibrotic conditions.

In Example 2, no evidence of plexiform changes in the vascular media, including pulmonary blood vessels, or fibroproliferative changes in any of the heart valves were observed in any group of dogs administered the inventive dosage forms.

In Example 3, mitral, aortic, pulmonic, and tricuspid regurgitation were assessed after administration of inventive dosage forms. All assessments of valve regurgitation were similar between the active group and the control group at baseline. Only minor and clinically insignificant changes in valve regurgitation were observed in the active group between baseline and week 26 and between baseline and week 52 of the open label study. In most cases, assessments were identical or slightly improved between these measures. These echocardiogram parameters were not indicative of a deleterious effect of valvular changes after 26 or 52 weeks of study. Only minor and clinically insignificant changes in lung diffusion were observed in the active group (that received inventive dosage forms) between baseline and week 26 and between baseline and week 52 of the open label study. Similarly, the mean changes in FEV₁ were minor and clinically insignificant. These pulmonary function parameters were not indicative of pulmonary fibrosis or degradation in lung function after 26 or 52 weeks of study.

Example 1 provides an illustrative method for making inventive compositions.

The invention will now be described in more detail.

### DEFINITIONS

All publications, patents and patent applications cited herein, whether supra or infra, are hereby incorporated by reference in their entirety for all purposes.

As used herein and in the appended claims, the singular forms “a,” “an,” and “the” include plural reference unless the context clearly dictates otherwise. Thus, for example, a reference to “a particle” includes a plurality of such particles, and a reference to “a carrier” is a reference to one or more carriers and equivalents thereof, and so forth.

“Administering” or “administration” means dosing a pharmacologically active material, such as DHE, to a subject in a manner that is pharmacologically useful.

“Cumulative drug substance particle size distribution” means, for a given DHE particle sample, a cumulative DHE particle size distribution curve with the volumetric median diameter plotted along the x-axis and cumulative volume percent of the DHE particles plotted along the y-axis. For such a curve, d10 is defined as the point on a cumulative volume percent distribution curve wherein 10% of the dihydroergotamine mesylate particles have a smaller volumetric median diameter; and d90 is defined as the point on a cumulative volume percent distribution curve wherein 90% of the dihydroergotamine mesylate particles have a smaller volumetric median diameter. In embodiments, the invention relates to DHE particles having a cumulative drug substance particle size distribution with d10>0.5 micron volumetric median diameter and d90<5.0 micron volumetric median diameter; preferably a cumulative drug substance particle size distribution with d10>0.7 micron volumetric median diameter and d90<4.0 micron volumetric median diameter. The volumetric diameter is measured by laser diffraction sizing, using for instance a Sympatec Helos (Princeton, N.J.) instrument.

“Dihydroergotamine mesylate” or “DHE” means the mesylate salt of the compound known generically as dihydroergotamine, which has a chemical structure of:

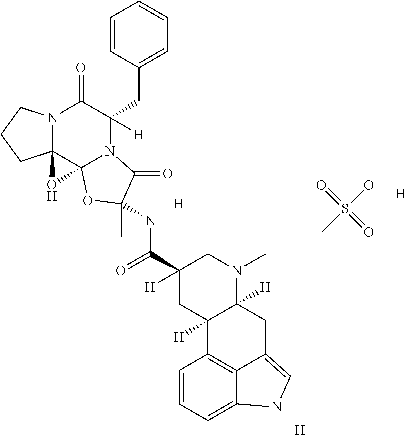

and common chemical name, ergotaman-3′,6′,18-trione,9,10-dihydro-12′-hydroxy-2′-methyl-5′-(phenylmethyl)-,(5′a)-, monomethanesulfonate. Its molecular weight is 679.80 and its empirical formula is C₃₃H₃₇N₅O₅.CH₄O₃S. In an embodiment, a dose of dihydroergotamine mesylate comprises between 0.1 mg to 4 mg of dihydroergotamine mesylate.

“Does not contribute to the development of” means that an incidence rate does not increase relative to control subjects.

“Subject” means a person or animal that is the object of treatment or observation.

“Migraine” has the meaning ascribed in International Classification of Headache Disorders 2^{nd} Edition in Cephalalgia 24: Suppl 1:9-160 (2004).

“Oral inhalation” means delivery of a drug, such as dihydroergotamine mesylate, to the lung via inhalation through the mouth.

### Metered Dose Inhalers and Formulation

A variety of dosage forms are useful in the practice of the invention, and are described in, for example, Published US Patent Application Number 2008/0118442. A few embodiments now will be discussed in more detail.

Metered dose inhalers (MDIs) conventionally have two components: a canister in which the drug particles are stored under pressure in a suspension or solution form, and a receptacle used to hold and actuate the canister. The canister may contain multiple doses of the formulation, although it is possible to have single dose canisters as well. The canister may include a valve, typically a metering valve, from which the contents of the canister may be discharged. Aerosolized drug is dispensed from the MDI by applying a force on the canister to push it into the receptacle, thereby opening the valve and causing drug particles to be conveyed from the valve through the receptacle outlet. Upon discharge from the canister, the drug particles are atomized, forming an aerosol. MDIs generally use propellants to pressurize the contents of the canister and to propel the drug particles out of the receptacle outlet. The propellant may take a variety of forms. For example, the propellant may be a compressed gas or a liquefied gas. Chlorofluorocarbons (CFC) were once commonly used as liquid propellants, but have now been banned. They have been replaced by the now widely accepted hydrofluroralkane (HFA) propellants, such as apaflurane and norflurane.

In some instances, a manual discharge of aerosolized drug must be coordinated with inhalation, so that the drug particles are entrained within the inspiratory air flow and conveyed to the lungs. In other instances, a breath-actuated trigger, such as that included in the Tempo® inhaler (MAP Pharmaceuticals, Mountain View, Calif.) may be employed that simultaneously discharges a dose of drug upon sensing inhalation, in other words, the device automatically discharges the drug aerosol when the user begins to inhale. These devices are known as breath-actuated metered dose inhalers (BApMDIs).

Typically, dosage forms according to the invention will be distributed, either to clinics, to physicians or to patients, in an administration kit, and the invention provides such a kit. Such kits comprise one or more of an administration device (e.g., inhalers, etc) and one or a plurality of doses or a reservoir or cache configured to deliver multiple doses of the composition as described above. In one embodiment, the dosage form is loaded with a DHE formulation. The kit can additionally comprise a carrier or diluent, a case, and instructions for employing the appropriate administration device. In some embodiments, an inhaler device is included. In one embodiment of this kit, the inhaler device is loaded with a reservoir containing a DHE formulation. In another embodiment the kit comprises one or more unit doses of the DHE formulation. In one embodiment, the inhaler device is a BApMDI such the TEMPO™ Inhaler.

A DHE powder useful in the present invention may be generated using supercritical fluid processes. Supercritical fluid processes offer significant advantages in the production of DHE particles for inhalation delivery. Importantly, supercritical fluid processes produce respirable particles of the desired size in a single step, eliminating the need for secondary processes to reduce particle size. Therefore, the respirable particle produced using supercritical fluid processes have reduced surface free energy, which results in a decreased cohesive forces and reduced agglomeration. The particles produced also exhibit uniform size distribution. In addition, the particles produced have smooth surfaces and reproducible crystal structures which also tend to reduce agglomeration.

Such supercritical fluid processes may include rapid expansion (RES), solution enhanced diffusion (SEDS), gas-anti solvent (GAS), supercritical antisolvent (SAS), precipitation from gas-saturated solution (PGSS), precipitation with compressed antisolvent (PCA), aerosol solvent extraction system (ASES), or any combinations of the foregoing. The technology underlying each of these supercritical fluid processes is well known in the art and will not be repeated in this disclosure. In one specific embodiment, the supercritical fluid process used is the SEDS method as described by Palakodaty et al. in US Application 2003 0109421.

In an embodiment, an inventive formulation can be made by suspending or dispersing the particles directly into a suspending media, such as a hydrofluoroalkane propellant. In one particular embodiment, the suspending media is the propellant. It may be desirable that the propellant not serve as a solvent to the DHE particles. Suitable propellants comprise hydrofluoroalkanes, preferably C₁₋₄ hydrofluoroalkanes, such as, but not limited to 1,1,1,2-tetrafluoroethane (HFA 134a or norflurane) and 1,1,1,2,3,3,3-heptafluoro-n-propane (HFA 227 apaflurane), either alone or in any combination. Carbon dioxide and alkanes, such as pentane, isopentane, butane, isobutane, propane and ethane, can also be used as propellants or blended with the C₁₋₄ hydrofluoroalkane propellants discussed above. In the case of blends, the propellant may contain from 0-25% of such carbon dioxide and 0-50% alkanes. In one embodiment, the DHE particulate dispersion is achieved without surfactants. In alternate embodiments, the DHE particulate dispersion may contain other excipients if desired, present in mass ratios to the DHE ranging from 0.001 to 10. Typical excipients might include surfactants such as oleates, stearates, myristates, alkylethers, alkylarylethers, sorbates and other surfactants used by those skilled in the art of formulating compounds for delivery by inhalation, or any combination of the foregoing. Specific surfactants include, but are not limited to, sorbitan monooleate sorbitan trioleate, isopropyl myristate, oleic acid, polysorbate 80, lecithin, methylparaben, polyethylene glycol, propylene glycol, povidone K-25, or propylparaben. The DHE particulate dispersion may also contain polar solvents in small amounts to aid in the solubilization of the surfactants, when used. Suitable polar compounds include 0₂₋₆ alcohols and polyols, such as ethanol, glycerol, isopropanol, polypropylene glycol and any combination of the foregoing. The polar compounds may be added at mass ratios to the propellant ranging from 0.0001% to 4%. Quantities of polar solvents in excess of 4% may react with the DHE or solubilize the DHE. In one particular embodiment, the polar compound is ethanol used at a mass ratio to the propellant from 0.0001 to 1%. No additional water or hydroxyl containing compounds are added to the DHE particle formulations other than is in equilibrium with pharmaceutically acceptable propellants and surfactants. The propellants and surfactants (if used) may be exposed to water of hydroxyl containing compounds prior to their use so that the water and hydroxyl containing compounds are at their equilibrium points. Other excipients might be used to improve surfactant solubility in the propellant or to prevent particle agglomeration or degradation including: lysine, glysine, lactose, mannitol, magnesium stearate, sodium citrate, edetate disodium.

In other embodiments, the inventive formulation comprises one or more excipients. In some embodiments, the inventive formulation comprises substantially no excipients.

Standard metering valves (such as from 3M, Valois, or Bespak) and canisters (such as from PressPart or 3M) can be utilized as is appropriate for the propellant/surfactant composition. Canister fill volumes from 2.0 ml to 17 ml may be utilized to achieve dose counts from one (1) to several hundred actuations. A dose counter with lockout mechanism can optionally be provided to limit the specific dose count irrespective of the fill volume. The total mass of DHE in the propellant suspension will typically be in the range of 0.100 mg to 2.000 mg of DHE per 100 mcL of propellant. An actuator with breath actuation can preferably be used to maximize inhalation coordination, but it is not mandatory to achieve therapeutic efficacy.

### Methods of Administration

The inventive formulations may be administered by oral inhalation, preferably using a metered dose inhaler, more preferably using a breath actuated metered dose inhaler. The recited formulations may be administered upon a subject's noticing the onset of a migraine, or at such other time as advised by a physician. Administration may take place 4 times/day, 3 times/day, 2 times/day, 1 time/day, 1 time/week, 1 time/month, or otherwise as appropriate. The dose will range from 0.05 mg to 4 mg of dihydroergotamine mesylate, preferably from 0.1 mg to 4 mg of dihydroergotamine mesylate. In some aspects, the dose of dihydroergotamine mesylate may range from 0.05 mg to 3.5 mg, 0.05 mg to 3.0 mg, 0.05 to 2.5 mg, 0.05 to 2 mg, 0.05 mg to 1.5 mg, 0.05 mg to 1.0 mg, or 0.05 to 0.1 mg. In other aspects, the dose of dihydroergotamine mesylate may be 0.05 mg, 0.1 mg, 0.5 mg, 1.0 mg, 1.5 mg, 2.0 mg, 2.5 mg, 3.0 mg, 3.5 mg, or 4 mg. In embodiments, a dose may comprise 1 or more inhalations, preferably 2 or more inhalations, and even more preferably 2 inhalations. In an embodiment, a nominal dose ranges from 0.1 mg to 4.0 mg of DHE. In another embodiment, an emitted dose ranges from 0.05 mg to 3.0 mg. In embodiments, a fine particle dose ranges from 0.05 mg to 2.0 mg of DHE, wherein the fine particle dose is defined as a dose of with a maximum particle size of 5.8 microns median mass aerodynamic diameter (MMAD.) The particle size may be determined by means known and standard in the art such as a cascade impactor, such as an Anderson Cascade Impactor also known as an “Apparatus 1” per USP 601.

The inventive compositions of matter can be used to treat a variety of conditions, including headaches. Headaches may comprise migraine, acute migraine, cluster headaches, adolescent migraine menstrual associated migraine, chronic migraine, medication overuse headache, and status migranosis.

As noted above, and elsewhere herein, while fibrosis is a potential adverse event associated with the administration of DHE through conventional routes, very low incidences of fibrosis have been noted in the study of administration of the inventive compositions of matter. In an embodiment, the invention relates to informing subjects that are being administered inventive compositions of matter or health care workers (such as doctors, nurses, or others who may have to administer or prescribe the inventive compositions of matter) that does not contribute to the development of a fibrotic condition in the subject, preferably wherein the fibrotic condition comprises pleural pulmonary fibrosis or valvulopathy.

### EXAMPLES

The invention will be more readily understood by reference to the following examples, which are included merely for purposes of illustration of certain aspects and embodiments of the present invention and not as limitations.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein.

Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

### Example 1

### Inventive Formulations

A mixing kettle is equipped with chilling jacket, a drug additional vessel (“DAV”), Lightning Mixer, and a 3 port cover and situated on a weight scale. The kettle is chilled to 0 Celsius, pressurized to approximately 500 millibars with dry Nitrogen, and then filled with approximately 80% of the total mass of the premixed blend of HFA227/HFA134a in 70/30 weight % ratio. The dry DHE particle powder is weighed into the DAV, and then the additional 20% of the propellant blend is pumped through the DAV, flushing the DHE powder into the vessel through a stainless steel tube under pressure of 500 millibars and at a temperature of approximately 0 Celsius. The force of the propellant impacting and flushing the drug powder charge into the kettle is sufficient to suspend/disperse the DHE particles in the propellant. When the propellant level in the kettle is sufficient to submerge the propeller of the Lightning Mixer, the mixer is energized to continuously stir the suspension at a medium speed. After mixing for 20 minutes following complete addition of the propellant, approximately 2.9 grams of the mixture corresponding to 17 milligrams of DHE in 2.2 milliliters of propellant is pumped through a Bespak 361 valve, which has been previously crimped on a 5.9 milliliter canister, filling approximately 50% of the valve canister assembly volume. The filled valve/canister assemblies are heat stress tested, valve discharge tested, weight checked and released for content and assay testing.

The filled valve/canister assemblies may then be mated into a TEMPO® breath actuated metered dose inhaler device (available from MAP Pharmaceuticals, Mountain View Calif.).

### Example 2

### Animal Trial of Dihydoergotamine Mesylate

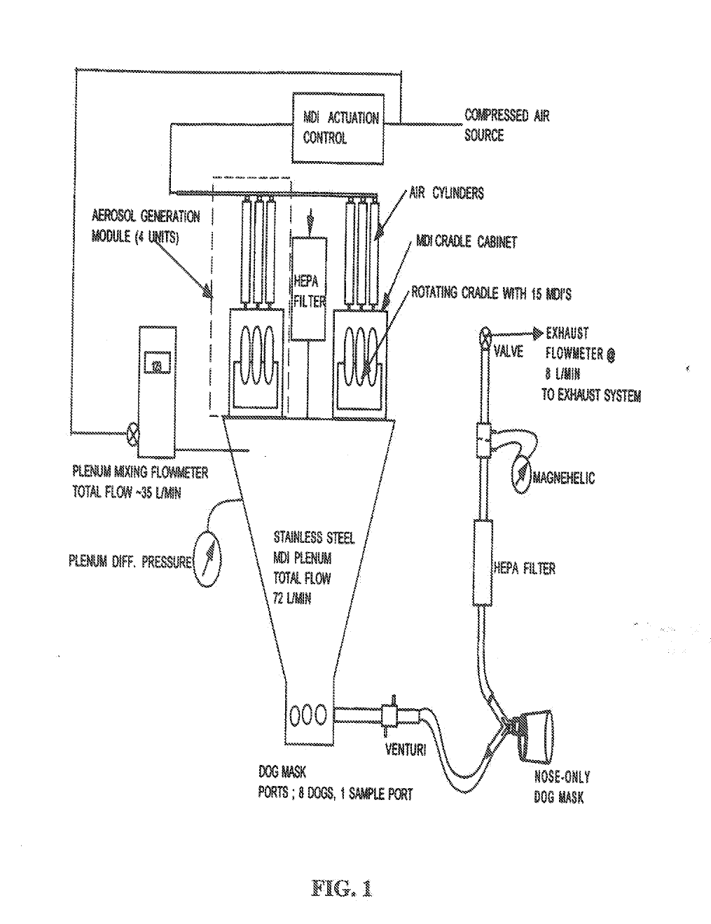

An inventive composition of matter was supplied in metered dose inhaler (MDI) canisters containing a stable concentration of DHE drug particles approximately 0.5 to 3 microns in diameter, suspended in a HFA227/HFA134a propellant blend vehicle. The formulation contained no other excipients or additives. Dosing was by nasal-only inhalation administered via an apparatus (shown in FIG. 1) including an aerosol generation module and a distribution plenum that could supply up to 8 dogs with a delivery tube/mask.

The generator was calibrated to produce a continuous stream of vehicle-only or DHE aerosol to each mask. By adjusting the ratio of airflow to DHE aerosol discharged from MDI units into the plenum, delivered drug concentrations could be adjusted to achieve target inhaled doses ranging from 0.0 milligram per kilogram (vehicle-only control) to approximately 1.08 milligram DH per kilogram (high dose) as shown in Table 1.

TABLE 1
Estimated Inhaled Doses of DHE

| | | | | | Approximate Multiples | |
|---|---|---|---|---|---|---|
| | | Target | | | in Humans | |
| Treat- | | Inhaled | Estimated Inhaled | | Daily | Thera- |
| ment | Dose | Dose | Dose (mg/kg) | | Systemic | peutic |
| Group | Level | (mg/kg) | Male | Female | Limit | Dose |
| 1 | Vehicle | 0 | 0 | 0 | 0 | 0 |
| 2 | Low | 0.056 | 0.049 | 0.041 | 2 | 3 |
| 3 | Mid | 0.224 | 0.135 | 0.172 | 5 | 11 |
| 4 | Mid-High | 0.54 | 0.42 | 0.46 | 15 | 31 |
| 5 | High | 1.08 | 0.74 | 0.91 | 29 | 58 |

After validation, a range of estimated inhaled doses was determined using cascade impaction and HPLC to characterize respirable aerosol delivered from each mask and adjusting for the minute volume of the male and female dogs (as shown in Table 1). The delivered high dose corresponded to approximately 29 times the highest daily systemic dose deemed safe in humans and was approximately 58 times higher than the currently effective 1 mg IV dose in humans. The low dose corresponded to approximately twice the highest daily systemic dose deemed safe in humans and was approximately 3 times higher than the currently therapeutically effective IV dose in humans.

Forty beagle dogs, 20 male and 20 female, were assigned to one of the 5 treatment groups. The dogs were dosed each day for 182 consecutive days. Dogs in Treatment Groups 2 to 5 received varying dose levels of DHE; dogs in Treatment Group 1 received HFA 227/134a vehicle only. Groups 1 to 4 were exposed to single daily inhalation exposures of 30 minutes; Group 5 was exposed twice daily for 30 minutes each session, the maximum feasible dose. Clinical endpoints used to evaluate the potential toxicity included: observations, physical examinations, body weights, food consumption, ophthalmic examinations, electrocardiogram analysis (including interval evaluation), and clinical and anatomical pathology including organ weights. Blood samples for toxicokinetic evaluation were obtained from all animals on Days 1, 28, 85, and 176.

Toxicokinetic results showed dose proportionality and rapid tmax similar to the IV reference. Signs of ergotism were reported at higher doses only. Abrasions and/or scabbing of the tips of the ear were noted in Groups 3, 4, and 5. Observations of emesis (vomiting) and excessive salivation during exposure were observed in Groups 3, 4, and 5. Emesis was observed in Group 5 immediately upon administration. Hematology, serum chemistry, and urinalysis remained within normal limits throughout.

No relevant changes in organ weights in Groups 2 to 5 were noted. No evidence of macro or microscopic changes was observed in the lungs in any dose group. Treatment related microscopic changes were minor and were noted in the respiratory nasal epithelium and to focal areas of the skin of the ears. All Group 5 animals were noted to have minimal nasal hyperplasia compared to no more than 2 animals in Groups 1 to 4. No evidence of plexiform changes in the vascular media, including pulmonary blood vessels, or fibroproliferative changes in any of the heart valves were observed in any group.

No significant respiratory tract toxicity was observed in dogs exposed to up to 1.08 mg/kg (more than 29 times the maximum safe daily IV human dose) of DHE per day for 6 months. The inventive composition of matter, when nasally administered, caused minor irritation of the anterior nasal epithelium, which manifested itself as increased nasal hyperplasia at the high dose. Clinical observations of expected systemic pharmacologic effects of the test article, such as emesis, excessive salivation during exposure, and abrasions and/or scabbing at the tip of the ears, were observed only at doses of 0.224 mg/kg (5 times maximum safe daily IV human dose) and higher.

### Example 3

### Human Trial of Dihydroergotamine Mesylate

The safety and efficacy of inhaled inventive DHE dosage forms were compared to placebo in a study in adult migraineurs for a single migraine at 2 hours and other specified time-points (from 10 minutes to 48 hours). 903 patients (450 on active drug and 453 on a placebo control) were enrolled in a randomized, double-blind, placebo-controlled, multi-center, parallel-group study of inventive DHE dosage forms in adult migraineurs. The inventive dosage forms used in the study were prepared generally according to the disclosure in Example 1.

The secondary objective of the study was to evaluate the safety of long-term exposure (up to 12 months of exposure) of the inventive DHE dosage form. 772 patients participated in the open label drug arm. In addition, 221 subjects participated in a control arm with no drug exposure (control subjects were not necessarily migraineurs).

The study population included adult migraineurs with a history of episodic, acute migraine, with or without aura (according to International Headache Society [IHS] criteria). The subjects must have been diagnosed with migraine for a minimum of 1 year prior to study entry and must have been diagnosed prior to the age of 50. Patients in the active drug arm received an emitted dose of 0.63 mg (1.0 mg nominal dose or 0.50 mg systemic equivalent dose) administered using the TEMPO® inhaler in two inhalations.

Patients participating in the open label were required to complete baseline cardiology (electrocardiography and echocardiography) and pulmonary function (both spirometry and lung diffusion) testing prior to any drug dosing (either the single dose in the double blind portion or the first dose in open label if they were not participants in the double blind portion). Patients completed electrocardiography and spirometry testing at each open label study visit, approximately every 8-12 weeks. Patients completed repeats of echocardiography and lung diffusion testing at approximately week 26 and week 52 of the study to be compared to the baseline tests. In addition, the control arm group completed baseline and repeat echocardiography and lung diffusion approximately 26 and 52 weeks following the baseline tests, similar to the open label group.

### Cardiovascular Safety Summary

Mitral, aortic, pulmonic, and tricuspid regurgitation were assessed. All assessments of valve regurgitation were similar between the active group and the control group at baseline. Only minor and clinically insignificant changes in valve regurgitation were observed in the active group between baseline and week 26 and between baseline and week 52 of the open label study. In most cases, assessments were identical or slightly improved between these measures. These echocardiogram parameters were not indicative of a deleterious effect of valvular changes after 26 or 52 weeks of study.

### Mitral Regurgitation

| | |
|---|---|
| Control | MAP0004 |

| | n | % | n | % |
|---|---|---|---|---|
| Baseline | | | | |
| Absent | 128 | 63.4 | 391 | 62.4 |
| Mild | 0 | 0.0 | 47 | 7.5 |
| Moderate | 10 | 5.0 | 1 | 0.2 |
| Trace | 64 | 31.7 | 188 | 30.0 |
| Total | 202 | | 627 | |
| Week 26 | | | | |
| Absent | 0 | — | 173 | 60.3 |
| Mild | 0 | — | 19 | 6.6 |
| Moderate | 0 | — | 1 | 0.3 |
| Trace | 0 | — | 94 | 32.8 |
| Total | 0 | | 287 | |
| Week 52 or ET | | | | |
| Absent | 0 | 0.0 | 54 | 58.1 |
| Mild | 0 | 0.0 | 6 | 6.5 |
| Moderate | 0 | 0.0 | 0 | 0.0 |
| Trace | 1 | 100.0 | 33 | 35.5 |
| Total | 1 | | 93 | |

### Aortic Regurgitation

| | |
|---|---|
| Control | MAP0004 |

| | n | % | n | % |
|---|---|---|---|---|
| Baseline | | | | |
| Absent | 194 | 96.0 | 589 | 93.9 |
| Mild | 2 | 1.0 | 10 | 1.6 |
| Moderate | 0 | 0.0 | 1 | 0.2 |
| Trace | 6 | 3.0 | 27 | 4.3 |
| Total | 202 | | 627 | |
| Week 26 | | | | |
| Absent | 0 | — | 265 | 92.3 |
| Mild | 0 | — | 4 | 1.4 |
| Moderate | 0 | — | 1 | 0.3 |
| Trace | 0 | — | 17 | 5.9 |
| Total | 0 | | 287 | |
| Week 52 or ET | | | | |
| Absent | 0 | 0.0 | 91 | 97.8 |
| Mild | 0 | 0.0 | 0 | 0.0 |
| Moderate | 0 | 0.0 | 0 | 0.0 |
| Trace | 1 | 100.0 | 2 | 2.2 |
| Total | 1 | | 93 | |

### Pulmonic Regurgitation

| | |
|---|---|
| Control | MAP0004 |

| | n | % | n | % |
|---|---|---|---|---|
| Baseline | | | | |
| Absent | 146 | 72.3 | 444 | 70.8 |
| N/A | 0 | 0.0 | 0 | 0.0 |
| Present | 56 | 27.7 | 183 | 29.2 |
| Total | 202 | | 627 | |
| Week 26 | | | | |
| Absent | 0 | — | 203 | 70.7 |
| N/A | 0 | — | 2 | 0.7 |
| Present | 0 | — | 82 | 28.6 |
| Total | 0 | | 287 | |
| Week 52 or ET | | | | |
| Absent | 1 | 100.0 | 74 | 79.6 |
| N/A | 0 | 0.0 | 1 | 1.1 |
| Present | 0 | 0.0 | 18 | 19.4 |
| Total | 1 | | 93 | |

### Tricuspid Regurgitation

| | |
|---|---|
| Control | MAP0004 |

| | n | % | n | % |
|---|---|---|---|---|
| Baseline | | | | |
| Absent | 0 | 0.0 | 1 | 0.2 |
| Mild | 14 | 6.9 | 56 | 8.9 |
| Moderate | 2 | 1.0 | 0 | 0.0 |
| N/A | 0 | 0.0 | 2 | 0.3 |
| Trace | 186 | 92.1 | 568 | 90.6 |
| Total | 202 | | 627 | |
| Week 26 | | | | |
| Absent | 0 | — | 2 | 0.7 |
| Mild | 0 | — | 25 | 8.7 |
| Moderate | 0 | — | 0 | 0.0 |
| N/A | 0 | — | 0 | |
| Trace | 0 | — | 260 | 90.6 |
| Total | 0 | | 287 | |
| Week 26 | | | | |
| Absent | 0 | 0.0 | 1 | 1.1 |
| Mild | 0 | 0.0 | 5 | 5.4 |
| Moderate | 0 | 0.0 | 0 | 0.0 |
| N/A | 0 | 0.0 | 0 | 0.0 |
| Trace | 1 | 100.0 | 87 | 93.5 |
| Total | 1 | | 93 | |

### Respiratory Safety Summary

Measurements of lung diffusion and Forced Expiratory Volume in 1 Second (FEV₁) were similar between the active group and the control group at baseline. Only minor and clinically insignificant changes in lung diffusion were observed in the active group between baseline and week 26 and between baseline and week 52 of the open label study. Similarly, the mean changes in FEV₁ were minor and clinically insignificant.

DLco (mL/min/mm Hg)

| | Control | MAP0004 |
|---|---|---|
| Baseline | | |
| N | 201 | 616 |
| Mean (SD) | 24.32 (5.191) | 23.27 (4.853) |
| Median | 23.08 | 22.53 |
| Min, Max | 14.40, 44.90 | 7.26, 48.83 |
| Week 26 | | |
| N | 2 | 260 |
| Mean (SD) | 25.73 (2.001) | 22.70 (4.825) |
| Median | 25.73 | 21.9 |
| Min, Max | 24.31, 27.14 | 10.90, 44.60 |
| Change from Baseline | | |
| to Week 26 | | |
| N | 2 | 255 |
| Mean (SD) | 2.07 (2.920) | −0.52 (2.627) |
| Median | 2.07 | −0.38 |
| Min, Max | 0.00, 4.14 | −14.56, 6.90 |
| Week 52 or ET | | |
| N | 1 | 89 |
| Mean (SD) | 26.60 (N/A) | 22.30 (4.531) |
| Median | 26.6 | 21.1 |
| Min, Max | 26.60, 26.60 | 14.40, 37.56 |
| Change from Baseline | | |
| to Week 52 or ET | | |
| N | — | 89 |
| Mean (SD) | — | −1.37 (3.114) |
| Median | — | −0.90 |
| Min, Max | — | −14.56, 5.00 |

### Mean Change from Baseline¹ in FEV₁ (L)

Mean Change from Baseline¹ in FEV₁ (L)

| | MAP0004 |
|---|---|
| | Total |
| Baseline-Actual | 2.84 |
| Week 0 (Visit 3) | 0.02 |
| Week 8 | 0.00 |
| Week 16 | 0.00 |
| Week 24 | 0.02 |
| Week 28 | −0.02 |
| Week 40 | −0.01 |
| Week 52 | −0.04 |
| End of Study² | 0.00 |

¹Baseline - Lower pre-randomization FEV₁ value
²End of Study - Measurement at Early Termination or Study Completion

These pulmonary function parameters were not indicative of pulmonary fibrosis or degradation in lung function after 26 or 52 weeks of study.

1-33. (canceled)

34. A dihydroergotamine (DHE) formulation for use in a metered dose inhaler, said formulation comprising one or more doses of the dihydroergotamine (DHE) formulation wherein:
said formulation consists of a hydrofluoroalkane propellant and dihydroergotamine mesylate particles, wherein the hydrofluoroalkane propellant consists of a mixture of 1,1,1,2-tetrafluoroethane and 1,1,1,2,3,3,3-heptafluoro-n-propane;
wherein each dose of the one or more doses of dihydroergotamine formulation consists of a fine particle dose ranging from 0.1 mg to 2.0 mg of dihydroergotamine mesylate particles, wherein the maximum particle size of the fine particle dose is about 5.8 microns mass median aerodynamic diameter; and
wherein the dihydroergotamine mesylate particles consists of a cumulative drug substance particle size distribution with d10>0.5 micron volumetric median diameter and d90<5.0 micron volumetric median diameter, wherein d10 is defined as the point on a cumulative volume percent distribution curve at which 10% of the dihydroergotamine mesylate particles have a smaller volumetric diameter and d90 is defined as the point on a cumulative volume percent distribution curve at which 90% of the dihydroergotamine mesylate particles have a smaller volumetric median diameter and volumetric diameter is as measured by laser diffraction sizing.

35. The dihydroergotamine (DHE) formulation of claim 1, wherein the mixture of 1,1,1,2-tetrafluoroethane and 1,1,1,2,3,3,3-heptafluoro-n-propane is in a 30:70 ratio.

36. The dihydroergotamine (DHE) formulation of claim 1, wherein the metered dose inhaler is a breath actuated metered dose inhaler.
